(19) United States
(12) Patent Application Publication
(10) Pub. No.: US 2019/0339241 A1
(43) Pub. Date: Nov. 7, 2019

(54) GAS DETECTION APPARATUS

(71) Applicant: ION SCIENCE LIMITED, Fowlmere, Cambridgeshire (GB)

(72) Inventors: Frank WIlliam Houlton DEAN, Cambridge (GB); Mark STOCKDALE, Cambridge (GB)

(73) Assignee: ION SCIENCE LIMITED, Fowlmere, Cambridgeshire (GB)

(21) Appl. No.: 16/475,307

(22) PCT Filed: Dec. 29, 2017
(86) PCT No.: PCT/GB2017/053905

§ 371 Date: Jul. 1, 2019

(30) Foreign Application Priority Data
Jan. 1, 2017 (GB) 1700019.1
Jul. 6, 2017 (GB) 1710891.1

(51) Int. Cl. G01N 33/00; G01N 30/28; G01N 27/70

(52) U.S. Cl. CPC G01N 33/0047; G01N 2030/884; G01N 27/70; G01N 30/28

(57) ABSTRACT

An apparatus detects a target gas in ambient air. The apparatus has a GC column, a sensor downstream of the GC column, a pump, a gas storage chamber and a pneumatic circuit. The pneumatic circuit has two states. In a first state, the pump draws in ambient air and supplies it to the gas storage chamber to store ambient air under pressure within the chamber, while trapping a sample of ambient air within the pneumatic circuit. In the second state, the gas storage chamber is connected to the GC column to cause pressurised air drawn from the storage chamber to act as a carrier gas to advance the trapped sample through the GC column and sensor. A filter is filters out any target gas present in the air entering into, or the air drawn from, the storage chamber, to avoid the presence of any target gas in the carrier gas.

### FIELD OF THE INVENTION

This invention relates to apparatus for the selective detection of the presence of a specific target gas in ambient air.

### BACKGROUND OF THE INVENTION

There is a need to detect and measure the presence of chemical compounds in air at variable concentration. Many such compounds are classed as volatile organic compounds, VOC's, which may be harmful to humans, animals and plant life, or may present a risk of fire or may be of other interest.

It is frequently of interest to search for the presence of gaseous species collectively, as unpolluted air contains very low concentrations of VOC's, and therefore the indication of any VOC's in air can often be attributed to a specific source. For example, a source of VOC leakage could be a spill of chemicals, a screened soil sample, a leak site in a chemical tank, or an accelerant in an arson attack. In such scenarios it is preferable for sensors and detectors of target analytes in air to provide a fast and quantitative measurement of their concentration, so that their provenance, in real time, can be ascertained. Sensors and detectors engaging photo-ionisation detection (PID), flame ionisation detection (FID), thermal conductivity detection (TCD) and amperometry are suitable for this purpose.

Specific VOCs, such as benzene, present a serious risk to health, making fast measurement of their concentration in ambient air particularly desirable. Such compounds may present themselves as mixtures with other VOC's which do not present such a danger to health. In detection of a specific VOC of concern, such as benzene, hereinafter for convenience called a target gas, it is well known that gas chromatography (GC) provides an effective method of pre-separation of the target gas from other detectable compounds in a test gas sample.

In gas chromatography, a small sample of gas to be analysed for the presence of a target gas is caused to enter a column that contains a medium, known as a stationary phase, onto which the gas is occasionally adsorbed and desorbed. For an extended time after introduction of the sample, a carrier gas devoid of the target gas is caused to flow through the GC column. By virtue of variable gas absorbency on the stationary phase, each gas constituent in the original sample gas emerges from the column at a characteristic time referred to as the gas elution time. By causing the gas exiting the chromatography column to be presented to a sensor, it is possible to detect the target gas from a prior knowledge of its elution time.

The combination of a GC column and a sensor, usually a PID sensor, has for many decades been used in analytical apparatus used in the laboratory. However, the technique, known as GC-PID, has hitherto only been used in bulky stationary apparatus.

It would be desirable to use the technique in a portable apparatus, that is to say a device sufficiently light and compact to be carried by an individual, so that personnel at risk may be warned when breathing air having a high concentration of the target gas. However, some of the processes commonly used in conventional GC-PID apparatus are incompatible with rendering the technology portable. In particular:
Many GC columns require a carrier gas such as helium or argon to draw the sample gas through a GC column. This is impractical in a portable apparatus.
Analytical GC-PID may require columns to be heated to high temperatures. This would present a substantial power burden to a small portable device.
Stationary apparatus have large GC columns with long elution times, often measured in the tens of minutes. As a result, the desired near immediacy of measurement in situ in a portable device would not be achieved.

From the above, it will be appreciated that, for it to be practicable, a portable apparatus requires a short column (for shorter elution times), incorporating a stationary phase that is stable on continuous exposure to air (to avoid the need for a supply of an inert carrier gas) and operable at a temperature that in only modestly above commonly encountered ambient temperatures (to reduce the power burden).

A GC column which is operable in air and meets the above requirements can be constructed from readily available materials and components. By way of an example, the column may comprise a metal tube having a length of 5 to 50 cm and a diameter of 1.2 mm diameter and filled with a diatomaceous earth support, such as Chromosorb® 120, which has been previously coated with a suitable stationary phase for separating a target VOC, such as benzene. Bis-cyanopropyl phases tend to be suitable for this purpose.

A further difficulty encountered in making a detecting apparatus portable does not relate to the performance of the GC column nor to the sensor, be it PID FID or TCD, but to the gas handling pneumatic circuit.

For successful operation of a GC-PID apparatus, a regular flow of gas is required through the GC column to set the background signal before introduction of the gas sample to be analysed. This conventionally calls for a pressure cylinder containing the carrier gas or a first pump that operates constantly. To introduce the gas sample into the GC column, a second pump, operating at higher pressure, is required along with various valves, conduits and connectors.

Components such as pumps and electrically controlled valves present several problems when designing a portable apparatus. In particular:
The components present a considerable power burden to a portable detection system.
Conventional valves may have substantial enclosed volumes which would prevent introduction of very small gas samples into the column.
Small commercially available high performance pumps are typically not configured to deliver the low gas flows demanded by miniaturised GC-PID detection. While this can be circumvented with bypass flows, it is difficult to balance pneumatically and reliably within portable detection systems.
The gas leaving the GC column and entering the detector is often at a pressure which is below that of ambient pressure. This is undesirable because any leak in the detector housing would cause ambient air to be mixed with gas leaving the GC column
The physical size of conventional pumps and pneumatic circuits make them unsuitable for a portable apparatus.

### SUMMARY OF THE INVENTION

According to a first aspect of the invention, there is provided an apparatus for detecting a target gas in ambient air, the apparatus comprising a GC column, a sensor located downstream of the GC column, a pump, a gas storage chamber and a pneumatic circuit that is operative in a first state to connect the pump to the gas storage chamber in order to store ambient air under pressure within the chamber, while trapping a sample of ambient air within the pneumatic circuit, and in a second state to connect the gas storage chamber to the GC column to cause pressurised air drawn from the storage chamber to act as a carrier gas to advance the trapped sample through the GC column and the sensor, wherein a filter is provided to filter out any target gas present in the air entering into, or the air drawn from, the storage chamber, so as to avoid the presence of any target gas in the carrier gas.

While air may be filtered to remove from it any target gas before it is pumped into the storage chamber, in a preferred embodiment the filter is positioned to filter air flowing from the storage chamber to the GC column.

In some embodiments, the gas storage chamber is a variable volume working chamber.

A variable volume working chamber may conveniently be formed by a bellows, but it is alternatively possible for the chamber to have a movable wall formed by a piston or a rolling diaphragm.

As well as removing target gas from the carrier gas, it is also desirable to filter out water vapour, by the use of a desiccant, in order to avoid problems caused by water condensation.

The design of the gas storage chamber should ensure that the carrier gas pressure, and the gas flow rate through the GB column, should be as constant and uniform as possible, at least for the period of time during which analysis of a sample by the GC column and the sensor is taking place.

The pneumatic circuit may suitably comprise a valve and conduits designed such that ambient air is supplied to the gas storage chamber through a conduit through which the filtered gas flows in the opposite direction towards the GC column, so that the ambient air trapped within the latter section of conduit may serve as the gas sample.

Conventional ambient air sampling systems, as described for example in WO2016/054585 rely on helium, or a gas other than ambient air, to serve as the carrier gas. Such systems employ a six port valve having a stator with six ports disposed in the same plane and spaced apart by 60°, and a rotor that has two positions. In one position, each of three ports is connected to the next port in the clockwise direction and in the second position each of the same three ports is connected to the next port in the counter-clockwise direction.

In some embodiments of the present invention, the pneumatic circuit employs a rotary 4-port two-position changeover valve, the rotor being formed with a conduit that connects two of the four ports in one position of the valve and the other two ports in the other position of the valve, the conduit being operative to trap a volume of gas to serve as the sample to be analysed.

A PID detector is suitable for use as a sensor but the type of sensor employed is not of critical importance to the invention, so long as it is capable of producing an electrical signal when the target gas exits the GC column.

### BRIEF DESCRIPTION OF THE DRAWINGS

The invention will now be described further, by way of example, with reference to accompanying drawings, in which:

FIGS. 1 and 2 are a schematic diagrams showing the manner in which 6-port valves are used in conventional GC-PID apparatus,

FIGS. 3 and 4 are schematic diagrams of an embodiment of the invention that uses a 4-port changeover valve,

FIG. 5 shows a schematic section through a 4-port rotary valve that may be used in the embodiment of FIG. 3,

FIG. 6 is a section through the valve of FIG. 5 in the plane A-A, and

FIG. 7 is a section through the valve of FIG. 5 in the plane B-B.

### DETAILED DESCRIPTION OF THE DRAWINGS

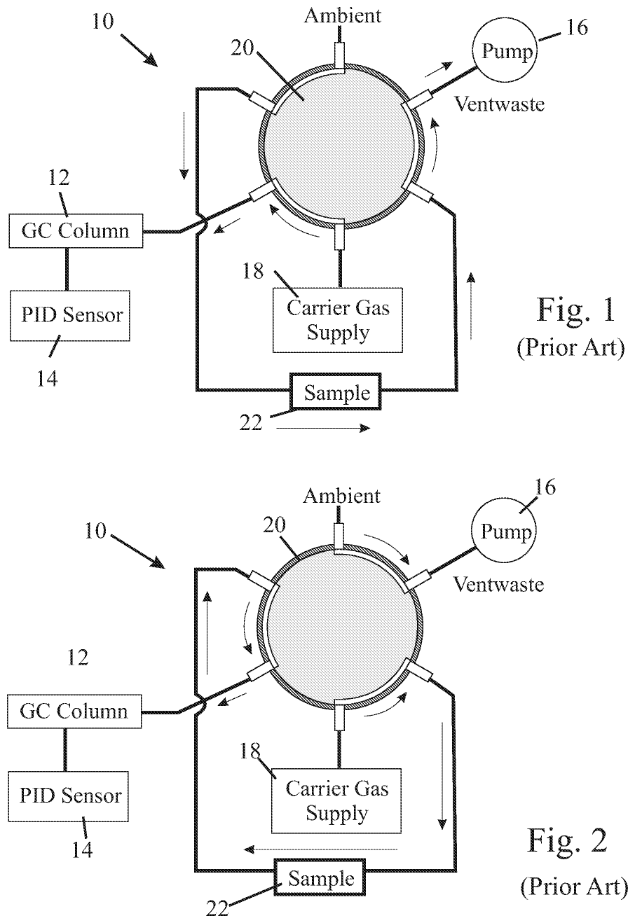

A conventional GC-PID apparatus 10 is shown in FIGS. 1 and 2. The apparatus comprises a GC column 12 followed by a PID sensor 14. The apparatus also comprises a gas pump 16, a source of a carrier gas 18 and a two position 6-port valve 20.

In a first position of the valve 20, shown in FIG. 1, a carrier gas, which is devoid of the target gas, fed under pressure from the supply 18 to the GC column 12 and flows out to ambient atmosphere through the PID sensor 14. The gas supply 18 may either be a pressure cylinder containing the carrier gas, or it may comprise a pump that pumps ambient air through a filter, such as an active carbon filter, into the GC column 12. At the same time, a separate pump 16 sucks ambient air into a loop that contains a reservoir 22 for the sample to be analysed.

To introduce the sample into the GC column, the valve 20 is rotated to the position shown in FIG. 2. In this position, carrier gas is supplied to the loop containing the sample reservoir 22, to transport the sample into the GC column 12 for analysis. During this time, the pump 16 merely draws in ambient air and discharges it as exhaust.

Such an apparatus is difficult to miniaturise for several reasons explained above. If the gas supply 18 is a pressure cylinder it would be cumbersome and heavy to permit the apparatus to operate continuously for an acceptable length of time. If it comprises a pump, then the need for both this pump and the pump 16 to operate continuously would place a heavy burden on the electrical power supply. The size of the sample reservoir and of the GC column result in long elution times, while in a portable apparatus it is desired to minimise the detection time.

A further disadvantage is that ambient air is sucked into the reservoir 22 and the sample resides in the reservoir 22 at sub-atmospheric pressure. If the sample remains under sub-ambient pressure on reaching the PID sensor 14, it creates a risk of ambient air being drawn into the sensor, if the sealing of the sensor is not perfect.

Existing valves of the type used in the pneumatic circuit as shown in FIGS. 1 and 2 have other inherent disadvantages, in that they have un-swept and contorted dead volumes that degrade the sample. Furthermore, they are costly, require a high current and may include component parts that interact with the sample.

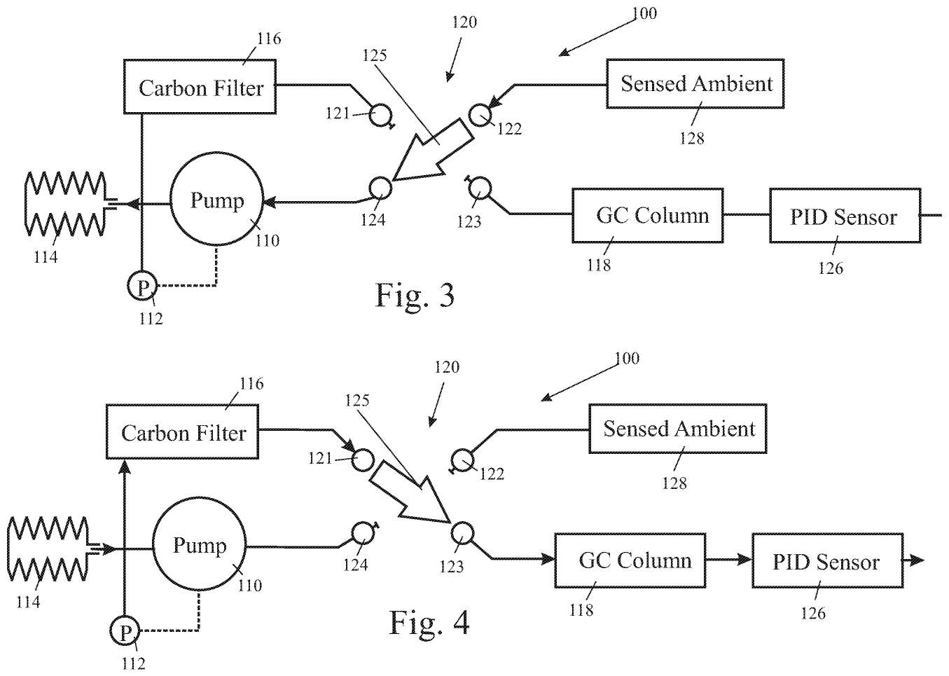

FIGS. 3 and 4 show an apparatus 100 embodying the present invention and using a 4-port changeover valve 120. The valve 120 has an internal conduit of fixed volume formed in its rotor and represented in the drawings by an arrow 125. The conduit in FIG. 3 connects the ports designated 122 and 124 and in FIG. 4 it connects the other two ports, designated 121 and 123.

Port 122 is connected to receive the ambient atmosphere 128 that is to be analysed. In the position of the valve 120 shown in FIG. 3A, a pump 110 connected to the port 124 draws the ambient atmosphere from the port 122 through the internal conduit 125 of the valve 120 and feeds the air under pressure into a variable volume storage chamber represented in the drawing by a bellows 114. A pressure sensor 112 sensing the output pressure of the pump 110 is used control the pump 110. The output of the pump 110 and the mouth of the bellows 114 are also connected by way of a carbon filter 116 to the port 121.

The port 123 of the valve 120 is connected to a GC column 118. Gas discharged from the GC column flows through a PID sensor 126 before being discharged to exhaust. In the position of the valve 120 shown in FIG. 3, the ports 121 and 123 are isolated so that no gas can reach the GC column 118 nor flow through the carbon filter 116.

To commence sample analysis, the rotor of the valve 120 is turned to the position shown in FIG. 4, in which ports 122 and 124 are isolated, while the internal conduit 125 of the valve 120 connects port 121 to port 123. In this position of the valve 120, pressurised ambient air stored in the bellows 114 flows through the carbon 116 filter to produce a carrier gas devoid of the target gas. The carrier gas flows through the internal conduit 125 to the GC column 118, sweeping ahead of it the fixed volume of ambient air trapped in the internal conduit 125, this volume being the sample to be analysed.

The gas sample now flows through the GC column 118 and its constituents leave the column 118 after different elution times. The target gas, if present, will reach the PID sensor 126 at a known time following the changeover of the position of the valve 120 and the strength of the output signal of the PID sensor 126 at this time will be indicative of the concentration of the target gas.

It will be appreciated that the carbon filter may be positioned between the output of the pump 110 and the input of the storage chamber 114, to remove target gas from the ambient air before it enters the storage chamber 114 instead cleaning the air after it has left the storage chamber, to allow it to serve as the carrier gas.

As well as filtering out the target gas, or VOC's generally, the filter 116, or a separate filter containing a desiccant, may be used to reduce the moisture content of the carrier gas to avoid condensation.

While it would be possible to use a fixed volume storage chamber 114, one having a variable volume is desirable as it helps keep to a minimum the volume of air that has to be pumped and filtered. If using a variable volume working chamber, a rolling diaphragm has been found to be the most efficient manner of achieving a movable wall.

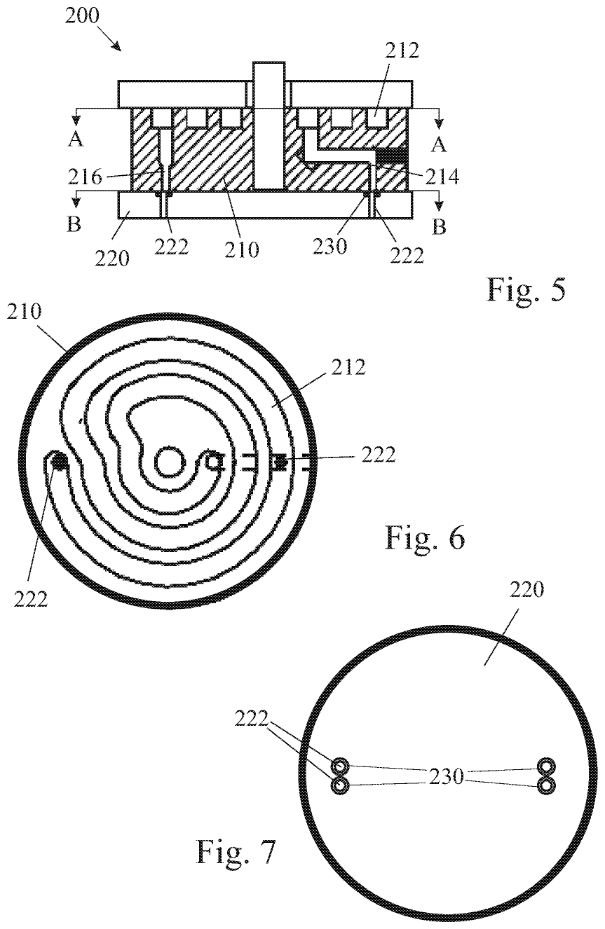

FIGS. 5 to 7 show a suitable construction of a 4-port valve 200. The valve has a rotor 210 into the top surface of which there is machined a spiral groove 212, best seen in FIG. 6, this being the internal conduit in which the sample is stored. The inner and outer ends of the spiral groove 212 are connected to bores 214, 216 that lie at equal distances from the axis of rotation of the rotor 210. The stator 220, as shown in FIG. 7, has four ports 222, each fitted with an O-ring 230, that can be selectively aligned, two at a time, with the bores 214 and 216 in the stator that connect to the ends of the spiral groove 212.

In operation, the apparatus starts in the position shown in FIG. 3 in which the bellows is charged until a desired pressure is sensed by the sensor 112. As an alternative, or in addition, to a pressure sensor 112, a mechanical sensor may be used to indicate when the variable volume working chamber 114 has been expanded to a desired size. Once the storage chamber 114 has been sufficiently charged, the pump 110 is switched off and the valve 120 moved to the position shown in FIG. 4. After completion of analysis of the sample stored in the internal conduit 125 of the valve 120, the valve 120 is returned to the position shown in FIG. 3 and the pump 110 is again operated for a brief interval, sufficient to recharge the storage chamber 114.

The valve shown in FIGS. 5 to 7 offers several important advantages. In particular, it will be noted that the ports 222 are arrange in two pairs with the ports of each pair located close to one another. Aside from allowing a quick changeover and requiring little movement of the rotor (thus minimising power consumption), the mouths of the bores 214 and 216 that communicate with the spiral groove containing the sample volume, are sealed by the O-rings 230 substantially the entire time during their transition between ports, thus avoiding any contamination of the trapped sample. Furthermore, those ports that are not in communication at any time with the internal conduit in the rotor of the valve are blocked off by the O-rings 230 sealing against the lower surface of the rotor 210 (as viewed in FIG. 5). Thus, in the position of the valve shown in FIG. 3, air cannot flow into, nor out of, the carbon filter through the port 121 and no gas can reach the GC column 118 from the port 123. During times that the bellows 114 is being recharged, the GC column remains filled with the carrier gas from the preceding sensing cycle and is therefore ready to receive the next sample to commence a new sensing cycle. In the position shown in FIG. 4, the blocking of the port 124 prevents the bellows from being discharged on account of reverse flow of air through the pump 110.

Despite the many advantages of the described and illustrated 4-port valve, it should be stressed that it does not form an essential part of the invention and may be replaced, for example, by electrostatic valves. Indeed, the entire pneumatic circuit using a 4-port valve is only given as an exemplary implementation of the invention.

There are several advantages presented by the disclosed embodiment of invention as compared with GC-PID apparatus provided by prior art. In particular:
Congruent with the requirements of a portable system, the apparatus employs only one pump which operates during only a fraction of the cycle time. The use of one pump reduces the size of the portable apparatus, and provides for relatively easy manufacture and service.
The variable volume storage chamber can be designed, such as by the use of a rolling diaphragm, to ensure that during the charging cycle the pump delivers a flow and pressure commensurate with its standard operation.
None of the flow is ‘wasted’ in bi-passes, thereby conserving energy.
The pneumatics are considerably simplified by the provision of a simple two stage process in which the gas sample is entrained.
The disposition of the gas sensor near the exhaust, avoids picking up detectable gas from leak sites.
A single absolute pressure sensor can be provided to maintain system fault diagnostics.
A smaller injection assembly is achieved.

1. Apparatus for detecting a target gas in ambient air, the apparatus comprising a GC column, a sensor located downstream of the GC column, a pump, a gas storage chamber and a pneumatic circuit incorporating a valve that is operative in a first state to connect the pump to the gas storage chamber in order to store ambient air under pressure within the chamber, while trapping a sample of ambient air within an internal conduit of the valve, and in a second state to connect the gas storage chamber to the GC column to cause pressurized air drawn from the storage chamber to act as a carrier gas to advance the trapped sample through the GC column and the sensor, wherein a filter is provided to filter out any target gas present in the air entering into, or the air drawn from, the storage chamber, so as to avoid the presence of any target gas in the carrier gas.

2. Apparatus as claimed in claim 1, wherein the filter is positioned in the path of the air drawn from the storage chamber.

3. Apparatus as claimed in claim 1, wherein the gas storage chamber is a variable volume working chamber.

4. Apparatus as claimed in claim 3, wherein the variable volume working chamber has a movable wall defined by a rolling diaphragm.

5. Apparatus as claimed in claim 1, wherein the valve is a rotary 4-port two-position changeover valve, the internal conduit being formed within the rotor to connect two of the four ports in one position of the valve and the other two ports in the other position of the valve.

6. Apparatus as claimed in claim 1, wherein the sensor is a PID sensor.

7. Apparatus as claimed in claim 1, wherein the filter, or an additional filter, serves to remove water vapour from the air serving as the carrier gas.

8. (canceled)
